(19) United States
(12) Patent Application Publication
(10) Pub. No.: US 2022/0348657 A1
(43) Pub. Date: Nov. 3, 2022

(54) BIOASSAY FOR T-CELL CO-STIMULATORY PROTEINS CONTAINING FC DOMAINS

(71) Applicant: FIVE PRIME THERAPEUTICS, INC., South San Francisco, CA (US)

(72) Inventors: Luis BORGES, San Mateo, CA (US); Artur KARASYOV, Oakland, CA (US); Zheng MENG, Burlingame, CA (US); Shawn RUSSELL, Waltham, MA (US); Nathan SALLEE, San Francisco, CA (US); Hongbing ZHANG, Albany, CA (US); Aileen ZHOU, Brisbane, CA (US)

(21) Appl. No.: 17/604,505

(22) PCT Filed: Apr. 17, 2020
(86) PCT No.: PCT/US2020/028715

§ 371 Date: Oct. 18, 2021

Related U.S. Application Data
(60) Provisional application No. 62/835,926, filed on Apr. 18, 2019.

(51) Int. Cl. C07K 16/28; C07K 14/705; G01N 33/554

(52) U.S. Cl. CPC C07K 16/2809; C07K 14/70532; G01N 33/554; C07K 2319/03; C07K 2319/30

(57) ABSTRACT

Provided herein are components (e.g., cells and soluble proteins), systems, and methods for assessing the biological activity of soluble proteins comprising an Fc domain and a CD28-binding domain (e.g., CD80 extracellular domain Fc fusion proteins).

### FIELD

The present disclosure relates generally to a compositions, systems, and methods for performing two-cell bioassays to assess the activity of a soluble fusion protein that contains an antibody Fc domain and a CD28-binding domain.

### BACKGROUND

Fusion proteins comprising the extracellular domain (ECD) of human cluster of differentiation 80 (CD80) and the fragment crystallizable (Fc) domain of human immunoglobulin G 1 (IgG1) have shown as promise as therapeutics for the treatment of cancers. See, for example, WO 2017/079117, which is herein incorporated by reference in its entirety. Conventional potency assays for biological therapeutics often involve two or more assays. A first assay may use a target-expressing cell line to evaluate the drug potency. This first assay focuses only on the target-binding portion of the therapeutic molecule. A second assay may use a binding assay and/or a cell-based assay to evaluate the Fc effector function of the drug. This second assay focuses only on the Fc portion of the therapeutic molecule. However, assays that evaluate target-binding and Fc portions of a therapeutic molecule separately do not mimic in vivo conditions. Moreover, such assays are not effective for determining the activity of therapeutic molecules that act in conjunction with T-cell co-stimulation.

Convenient and robust methods for measuring the effectiveness of therapeutics that target T-cell co-stimulation pathways are needed.

### BRIEF SUMMARY OF THE INVENTION

Provided herein are two-cell assays for determining the activity of soluble proteins comprising an Fc domain and a CD28-binding domain. As demonstrated herein, such assays are advantageous in engaging the two different functions of the soluble protein, i.e., the Fc receptor-binding function and the CD28-binding function, in one assay. Such assays are also advantageous in using a second stimulator to enhance co-signaling activation.

Provided herein is an engineered activator cell comprising (i) a T cell receptor (TCR) complex activator and (ii) and an Fc-gamma receptor or an Fc-binding fragment thereof.

In one instance, the TCR complex activator is an anti-CD3 antibody, an antigen-binding fragment thereof, or major histocompatibility complex (MHC).

In one instance, the TCR complex activator is an anti-CD3 antibody or antigen-binding fragment thereof. In one instance, the anti-CD3 antibody or antigen-binding fragment thereof binds to the epsilon subunit of CD3. In one instance, the anti-CD3 antibody or antigen-binding fragment thereof is capable of inducing T cell activation. In one instance, the anti-CD3 antibody or antigen-binding fragment thereof is OKT3 or an antigen-binding fragment thereof. In one instance, the anti-CD3 antibody or antigen-binding fragment thereof comprises the amino acid sequence set forth in SEQ ID NO:6. In one instance, the anti-CD3 antibody or antigen-binding fragment thereof is produced from a protein comprising the amino acid sequence set forth in SEQ ID NO:8.

In one instance, the activator cell comprises a nucleic acid encoding the TCR complex activator, wherein the nucleic acid encoding the TCR complex activator is operably linked to a CAG promoter.

In one instance, the Fc-gamma receptor or Fc-binding fragment thereof is an IgG receptor or Fc-binding fragment thereof. In one instance, the Fc-gamma receptor or Fc-binding fragment thereof is CD64 or an Fc-binding fragment thereof In one instance, the Fc-gamma receptor or Fc-binding fragment thereof comprises the amino acid sequence set forth in SEQ ID NO:7. In one instance, the Fc-gamma receptor or Fc-binding fragment thereof is produced from a protein comprising the amino acid sequence set forth in SEQ ID NO:9. In one instance, the activator cell comprises a nucleic acid encoding the the Fc-gamma receptor or Fc-binding fragment thereof, wherein the nucleic acid encoding the Fc-gamma receptor or Fc-binding fragment thereof is operably linked to a CAG promoter.

In one instance, the activator cell is a HEK293 cell.

In one instance, the activator cell is stable for at least 1 month. In one instance, the activator cell is stable for at least 2 months. In one instance, the activator cell is stable for at least 3 months.

Provided herein is a composition comprising a plurality of any activator cells provided herein.

In one instance, the activator cells have at least 90% viability. In one instance, the activator cells have at least 94% viability. In one instance, the activator cells have about 94% to about 97% viability.

In one instance, the composition is frozen. In one instance, the composition is thawed after previously being frozen.

Provided herein is a cell-based assay system comprising any activator cell provided herein and an effector cell, wherein the effector cell comprises a TCR complex and CD28.

In one instance, upon interaction of a soluble protein comprising an Fc domain and a CD28-binding domain with (i) the Fc-gamma receptor or Fc-binding fragment thereof on the activator cell and (ii) the CD28 on the effector cell, the TCR complex activator activates the TCR complex and the CD28-binding domain activates the CD28 signaling pathway.

In one instance, the effector cell is a T-cell. In one instance, the T-cell is a Jurkat,

HuT-78, CEM, Molt-4, or primary T-cell. In one instance, the effector cell is a Jurkat T-cell.

In one instance, the effector cell further comprises a reporter of TCR complex and

CD28 activation.

In one instance, the effector cell further comprises a reporter gene, the expression of which is under the control of a TCR-pathway-dependent promoter. In one instance, the TCR-pathway-dependent promoter is an IL2 promoter. In one instance, the reporter gene comprises a nucleic acid sequence encoding a bioluminescent protein. In one instance, the reporter gene comprises a nucleic acid sequence encoding a luciferase, a beta lactamase, CAT, SEAP, a fluorescent protein, or a quantifiable gene product. In one instance, the reporter gene comprises a nucleic acid sequence encoding a luciferase.

In one instance, the system comprises about 40,000 to about 55,000 activator cells. In one instance, the system comprises about 40,000 to about 50,000 activator cells. In one instance, the system comprises about 50,000 activator cells.

In one instance, the ratio of activator cells to effector cells is about 1:4 to about 1:1.

In one instance, the system further comprises at least one soluble protein comprising an Fc domain and a CD28-binding domain.

In one instance, the system comprises about 0.4 million to about 0.55 million activator cells/mL. In one instance, the system comprises about 0.4 million to about 0.5 million activator cells/mL. In one instance, the system comprises about 0.5 million activator cells/mL.

In one instance, the system comprises about 50,000 to about 200,000 effector cells. In one instance, the system comprises about 100,000 effector cells.

In one instance, the system comprises about 0.5 million to about 2 million effector cells/mL. In one instance, the system comprises about 1 million effector cells/mL.

In one instance, the ratio of activator cells to effector cells in the system is about 1:4 to about 1:1.

In one instance, the system further comprises at least one soluble protein comprising an Fc domain and a CD28-binding domain. In one instance, the soluble protein is present at a concentration of about 1 ng/mL to about 5000 ng/mL. In one instance, the soluble protein is present at a concentration of about 1 ng/mL to about 3000 ng/mL

Provided herein is a system comprising any activator cell provided herein and at least one soluble protein comprising an Fc domain and a CD28-binding domain. In one instance, the soluble protein is present at a concentration of about 1 ng/mL to about 5000 ng/mL. In one instance, the soluble protein is present at a concentration of about 1 ng/mL to about 3000 ng/mL.

Provided herein is a method comprising (a) incubating the activator cell and the effector cell in any system provided herein and a soluble protein comprising an Fc domain and a CD28-binding domain and (b) detecting a signal from the reporter.

Provided herein is a method comprising (a) serially diluting a soluble protein comprising an Fc domain and a CD28-binding domain, (b) incubating the activator cell and the effector cell in any system provided herein with the serially diluted soluble protein of (a), and (c) detecting a signal from the reporter.

In one instance, the method further comprises comparing the signal from the reporter to the signal generated by a reference standard.

Provided herein is a method comprising (a) incubating the activator cell and the effector cell in any system provided herein, (b) detecting a signal from the reporter in the effector cell of (a), (c) incubating the activator cell and the effector cell in any system provided herein with a soluble protein comprising an Fc receptor and a CD28-binding domain, (d) detecting a signal from the reporter in the effector cell of (c), and (e) comparing the signal of step (b) with the signal of step (d), wherein a gain of signal from step (b) to step (d) indicates biological activity of the soluble protein.

In one instance, the amount of signal is proportional to the activity of the soluble protein present.

In one instance, the incubating is for 18-22 hours.

In one instance, the method further comprises thawing the activator cells before the incubation.

In one instance, the Fc domain is a human IgG1 Fc domain. In one instance, the

Fc domain comprises the amino acid sequence set forth in SEQ ID NO:3.

In one instance, the CD28-binding domain comprises CD80 or a fragment thereof,

CD86 or a fragment thereof, or an anti-CD28 antibody or fragment thereof. In one instance, the CD28-binding domain comprises the extracellular domain of CD80. In one instance, the extracellular domain of CD80 comprises the amino acid sequence set forth in SEQ ID NO:1.

In one instance, the soluble protein comprises the amino acid sequence set forth in

SEQ ID NO:5.

In one instance, the at least one soluble protein further comprises sialic acid (SA).

In one instance, the soluble protein comprises 15-60 moles of SA per mole protein. In one instance, the soluble protein comprises 15-40 moles of SA per mole protein. In one instance, the soluble protein comprises 15-30 moles of SA per mole protein. In one instance, the soluble protein comprises 20-60 moles of SA per mole protein. In one instance, the soluble protein comprises 20-30 moles of SA per mole protein.

### BRIEF DESCRIPTION OF THE DRAWINGS/FIGURES

FIG. 1 provides a schematic of an exemplary two-cell bioassay using an activator cell and an effector cell to measure the activity of a soluble protein, e.g., a CD80 extracellular domain Fc fusion protein. The activity of the fusion protein is quantified through the measurement of signal from a luciferase reporter construct and the relative potency is measured by comparison to a reference standard. (See Example 1.)

FIG. 2 shows the separation of activator cells with low, medium, and high levels of CD64 expression. (See Example 2.)

FIG. 3 shows the effects of CD64 levels on assay performance. (See Example 2.)

FIG. 4 shows the separation of activator cells with low, medium, and high levels of OKT3 expression. (See Example 2.)

FIG. 5 shows the effects of OKT3 levels on assay performance. (See Example 2.)

FIG. 6 shows the effect of cell seeding density on assay performance. (See Example 3.)

FIG. 7 shows that a clonal activator cell line demonstrates stable assay performance for up to 3 months. “P21” refers to passage 21. (See Example 4.)

FIG. 8 shows that activator cells can be used after they are frozen and thawed. (See Example 5.)

### DETAILED DESCRIPTION OF THE INVENTION

### I. Definitions

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular.

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The terms “polypeptide,” “peptide,” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present invention, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

A “fusion molecule” as used herein refers to a molecule composed of two or more different molecules that do not occur together in nature being covalently or noncovalently joined to form a new molecule. For example, fusion molecules may be comprised of a polypeptide and a polymer such as PEG, or of two different polypeptides. A “fusion protein” refers to a fusion molecule composed of two or more polypeptides that do not occur in a single molecule in nature.

A “CD80 extracellular domain” or “CD80 ECD” refers to an extracellular domain polypeptide of CD80, including natural and engineered variants thereof. A CD80 ECD can, for example, comprise, consist essentially of, or consist of the amino acid sequence set forth in SEQ ID NO:1 or 2. A “CD80 ECD fusion molecule” refers to a molecule comprising a CD80 ECD and a fusion partner. The fusion partner may be covalently attached, for example, to the N- or C-terminal of the CD80 ECD or at an internal location. A “CD80 ECD fusion protein” is a CD80 ECD fusion molecule comprising a CD80 ECD and another polypeptide that is not naturally associated with the CD80 ECD, such as an Fc domain. A CD80 ECD fusion protein can, for example, comprise, consist essentially of, or consist of the amino acid sequence set forth in SEQ ID NO: 4 or 5.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The term “isolated” as used herein refers to a molecule that has been separated from at least some of the components with which it is typically found in nature. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The term “reporter” is used herein to describe a molecular entity, a property and/or activity of which (e.g., concentration, amount, expression, localization, interactions such as protein-protein interactions, etc.) can be detected and correlated with a pathway, process, characteristic and/or activity of a system containing the reporter (e.g., cell, cell lysate, in vitro system, organism, in vivo system, etc.). A “reporter” may be an intrinsic (e.g., endogenous) element of the system, or an exogenous (e.g., artificial) element engineered or introduced into the system, that exhibits a detectable property or activity correlated with a pathway, process, characteristic or activity (e.g., gene expression) of the system or a component within the system. Suitable reporters include, but are not limited to: intrinsic genes or proteins or exogenous genes or proteins, the latter including, e.g., luciferases, beta lactamases, CAT, SEAP, fluorescent proteins, etc.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

As used herein, the terms “about” and “approximately,” when used to modify a numeric value or numeric range, indicate that deviations of 5% to 10% above and 5% to 10% below the value or range remain within the intended meaning of the recited value or range.

Any components, systems, or methods provided herein can be combined with one or more of any of the other components, systems, and methods provided herein.

### II. Activator Cells

Engineered activator cells are provided herein. In certain embodiments, an activator cell is engineered to express a T cell receptor (TCR) complex activator and an Fc-gamma receptor or an Fc-binding fragment thereof on its surface. The TCR complex activator can engage a TCR complex on the surface of an effector cell, and the Fc-gamma receptor or Fc-binding fragment thereof can engage CD28, also on the surface of the effector cell, in the presence of a soluble molecule that binds to both the Fc-gamma receptor or the Fc-binding fragment thereof (on the activator cell) and CD28 (on the effector cell). The soluble molecule can thus create a “bridge” between the activator cell and the effector cell, while the TCR complex activator on the activator cell can engage directly with the TCR complex on the effector cell. These interactions allow the activator cell to activate TCR-dependent signaling in the effector cell, which can be detected by measuring expression of certain endogenous genes or reporter genes, as described below.

The TCR complex activator can be any protein or protein complex capable of inducing T cell activation (e.g., in combination with a co-stimulatory signal) via the TCR complex. As explained in additional detail in Section III below, a TCR complex can comprise a T-cell receptor and CD3. The T-cell receptor can comprise a heterodimer, e.g., a heterodimer of TCR-alpha and TCR-beta chains or a heterodimer of TCR-gamma and TCR-delta chains. The T-cell receptor in the TCR complex can be associated (e.g., noncovalently) with a multisubunit CD3 signaling apparatus. The CD3 signaling apparatus can comprise CD3ϵγ and CD3ϵδ heterodimers and a CD3ζζ homodimer.

A TCR complex activator is a protein or complex of proteins that binds to one more components of the TCR complex and results in signaling of the TCR complex. A TCR complex activator can be, for example, an anti-CD3 (e.g., CD3ϵ) antibody or antigen-binding fragment thereof. The antigen-binding fragment can be, for example, an scFv. Other examples of TCR complex activators include superantigens, anti-TCR antibodies, anti-CD2 antibodies, anti-CD4 antibodies, PHA, major histocompatibility complex (MHC) and cognate peptides, and Con A.

In certain instances, the TCR complex activator is an anti-CD3 antibody or antigen-binding fragment thereof that binds to the epsilon subunit of CD3 (i.e., CD3ϵ). The TCR complex activator can be, for example, OKT3 or an antigen-binding fragment thereof, such as an scFv. OKT3 is a murine monoclonal anti-CD3 antibody of the immunoglobulin IgG2a isotype. It is available, for example, from BioLegend® as catalog #317301.

In certain instances, the TCR complex activator is an OKT3 scFv that comprises, consists essentially of, or consists of the amino acid sequence set forth in SEQ ID NO:6. In certain instances, the OKT3 scFv is initially expressed as a protein comprising SEQ ID NO:8 and then cleaved to produce a mature protein. In certain embodiments, the mature protein comprises or consists of the amino acid sequence of SEQ ID NO:6.

In certain instances, the TCR complex activator is expressed on the activator cell at a level that is sufficiently low (e.g., as determined by FACS) to minimize background signal.

The Fc-gamma receptor or Fc-binding fragment thereof can be an IgG receptor or fragment thereof such as FcγI (CD64), FcγII, FcγIII, or an Fc-binding fragment thereof. In certain instances the Fc-gamma receptor or Fc-binding fragment thereof comprises one immunoglobulin-like domain. In certain instances the Fc-gamma receptor or Fc-binding fragment thereof comprises at least one immunoglobulin-like domain. In certain instances the Fc-gamma receptor or Fc-binding fragment thereof comprises two immunoglobulin-like domains. In certain instances the Fc-gamma receptor or Fc-binding fragment thereof comprises at least two immunoglobulin-like domains. In certain instances the Fc-gamma receptor or Fc-binding fragment thereof comprises three immunoglobulin-like domains.

In certain instances, an Fc-binding fragment of an Fc-gamma receptor does not contain the intracellular domain of the Fc-gamma receptor. The absence of the intracellular domain can avoid triggering signaling within the activator cell that would otherwise interfere with interpretation of assay results.

In certain instances, an Fc-gamma receptor is engineered to comprise an Fc-binding fragment of an Fc-gamma receptor fused to a cell surface localization domain, e.g., a transmembrane domain. The transmembrane can be any heterologous transmembrane domain that results in cell surface localization of the Fc-binding fragment. In certain embodiments, the heterologous transmembrane domain is the transmembrane domain of CD32. In certain embodiments, the Fc-binding fragment is an Fc-binding fragment of CD64, and the heterologous transmembrane domain is the transmembrane domain of CD32. In such embodiments, the Fc-gamma receptor or Fc-binding fragment thereof comprises, consists essentially of, or consists of the amino acid sequence set forth in SEQ ID NO:7. In certain instances, the Fc-gamma receptor or Fc-binding fragment thereof is initially expressed as a protein comprising SEQ ID NO:9 and then cleaved to produce a mature protein. In certain embodiments, the mature protein comprises or consists of the amino acid sequence of SEQ ID NO:7.

In certain instances, the Fc-gamma receptor or Fc-binding fragment thereof is expressed on the activator cell at a level that is sufficiently high (e.g., as determined by FACS) to optimize the signal induced by a soluble protein that engages both the Fc-gamma receptor or Fc-binding fragment thereof and CD28 on the effector cell (e.g., a CD80 extracellular domain Fc fusion protein).

The activator cell can be engineered from an immune-derived cell or a non-immune-derived cell. Exemplary immune-derived cells include K562 cells, and exemplary non-immune-derived cells include HEK293 cells. “HEK293 cells” include parental HEK293 cells as well as derivatives thereof, such as HEK293T/17 cells (ATCC No. CRL-11268, and as further described in Pear et al. (1993) “Production of High-Titer Helper-Free Retroviruses by Transient Transfection,” Proc. Natl. Acad. Sci. USA 90:8392-8396).

The activator cell be a stable cell line, e.g., stable for at least 1 month, for at least 2 months, for at least 3 months, or more. The activator cell can be active after freezing and thawing.

Also provided herein are compositions comprising a plurality of activator cells. In certain embodiments, the activator cells in the composition are from a single clonal cell line. In certain embodiments, the composition comprises a plurality of activator cells that differ in their expression levels of the TCR complex activator (e.g., an anti-CD3 or OKT3 scFv) by at least 10-fold or 100-fold as determined, e.g., by FACS. In certain embodiments, the composition comprises a plurality of activator cells that that differ in their expression levels of an Fc-gamma receptor or Fc-binding fragment thereof (e.g., CD64) by at least 10-fold or 100-fold as determined, e.g., by FACS. In certain embodiments, the composition comprises a plurality of activator cells that differ in their expression levels of the TCR complex activator (e.g., an anti-CD3 or OKT3 scFv) by at least 10-fold or 100-fold as determined, e.g., by FACS, and that differ in their expression levels of the Fc-gamma receptor or Fc-binding fragment thereof (e.g., CD64) by at least 10-fold or 100-fold as determined, e.g., by FACS. A composition comprising a plurality of activator cells can be frozen or can be thawed from a frozen composition. A composition comprising a plurality of activator cells can be a cell culture.

In certain instances, the activator cells in the composition have at least 90%, at least 91%, at least 92%, at least 93%, or at least 94% viability. In certain instances, the activator cells have about 94% to about 97% viability. Viability can be determined for example, by trypan blue exclusion in which dead cells take up the trypan blue cells, whereas live cells do not (e.g., using Vi-CELL (Beckman Coulter) or a hemocytometer).

Also provided are containers, such as vials, comprising about 5×10⁶-5×10⁷ activator cells; about 5×10⁶-1×10⁷ activator cells; or about 10⁷ activator cells, as provided herein. The cells may be frozen or thawed in the vial.

Also provided herein are methods of making activator cells, the method comprising transfecting cells with nucleic acid encoding a TCR complex activator and a nucleic acid encoding a Fc-gamma receptor or Fc binding fragment thereof. The nucleic acid encoding a TCR complex activator can be operably linked to a promoter, e.g., a CMV early enhancer/chicken β actin (CAG) promoter. The nucleic acid encoding the Fc-gamma receptor or Fc binding fragment thereof can be operably linked to a promoter, e.g., a CAG promoter. In certain instances, the nucleic acid encoding a TCR complex activator is operably linked to a CAG promoter, and the nucleic acid encoding the Fc-gamma receptor or Fc binding fragment thereof is operably linked to a CAG promoter.

The nucleic acid encoding the TCR complex activator and the nucleic acid encoding the Fc-gamma receptor or Fc binding fragment thereof can be in the same polynucleotide (e.g., vector) or can be in separate polynucleotides (e.g., separate vectors). When separate polynucleotides are used, the transfecting may occur concurrently or sequentially in either order. The cells can be, for example, HEK293 cells. The method can further comprise selecting transfected cells that express the TCR complex activator and the Fc-gamma receptor or Fc binding fragment thereof. The method can further comprise selecting cells that express the TCR complex activator at low levels, i.e., within the 5^{th}, 10^{th}, 15^{th} or 20^{th} percentile for expression levels in the transfected cell population. The method can further comprise selecting a single cell comprising the nucleic acid encoding the TCR complex activator and the nucleic acid encoding the Fc-gamma receptor or Fc binding fragment thereof, and optionally expanding the single cell to create a clonal population of activator cells.

### III. Effector Cells

The activator cells provided herein can be used along with effector cells to determine the activity of a soluble protein (e.g., a CD80 extracellular domain Fc fusion protein). An effector cell comprises a TCR complex, CD28, and a reporter. Activation of the effector cell is initiated by engagement of both the TCR complex and the CD28. In T cells, engagement of both the TCR complex and CD28 can lead to activation of ERK/JNK and IκB kinase (IKK), which in turn regulate transcriptional activation of AP-1 and NF-κB pathways, respectively. The IL-2 promoter contains DNA binding sites for AP-1 and NF-κB. Therefore, engagement of both the TCR complex and CD28 results in IL-2 production, which is commonly used as a functional readout for T cell activation. As a proxy for IL-2 production, a reporter construct may be used that expresses a reporter molecule under control of the IL-2 promoter, which refers to a fully intact IL-2 promoter or a functional fragment thereof.

As provided herein, expression of the TCR complex and CD28 on the effector cell allows the effector cell to activate TCR-dependent signaling in the presence of an activator cell provided herein, which expresses a TCR-complex activator, and a soluble molecule that bridges the Fc-gamma receptor or the Fc-binding fragment thereof on the activator cell to the CD28 on the effector cell. The TCR-dependent signaling in the effector cell results in a detectable signal from the reporter and requires engagement of both the TCR complex and the CD28. Activation of the TCR complex and CD28 can activate the reporter and/or expression of the reporter, thereby detectably signaling effective interaction of the soluble protein with the Fc-gamma receptor or the Fc-binding fragment thereof and the CD28.

Exemplary effector cells are provided in WO 2016/0181854, which is herein incorporated by reference in its entirety. In certain instances, the effector cell is the “TCR/CD3 Effector Cell” of Promega's T Cell Activation Bioassay (catalog #J1651).

A TCR complex can comprise a T-cell receptor and CD3. In general, the T-cell receptor is capable of ligand recognition, e.g., recognition of peptide antigens in the context of MHC molecules. CD3 is capable of signaling in response to ligand recognition, and signaling by CD3 can also be activated by anti-CD3 antibodies and activating fragments thereof. The T-cell receptor can comprise a heterodimer, e.g., a heterodimer of TCR-alpha and TCR-beta chains or a heterodimer of TCR-gamma and TCR-delta chains. In certain instances, the T-cell receptor comprises a heterodimer of TCR-alpha and TCR-beta chains. The T-cell receptor in the TCR complex can be associated (e.g., noncovalently) with a multisubunit CD3 signaling apparatus. The CD3 signaling apparatus can comprise CD3ϵδ and CD3ϵδ heterodimers and a CD3ζζ homodimer. The TCR complex can comprise a 1:1:1:1 stoichiometry for the TCR (e.g., alpha-beta heterodimer):CD3ϵγ:CD3ϵδ:CD3ζζ dimers.

An effector cell can be an immune cell that endogenously expresses a TCR complex. An effector cell can be an immune cell that endogenously expresses CD28. An effector cell can be an immune cell that endogenously expresses a TCR complex and CD28. An effector cell can be an immune cell that is transfected to express any TCR complex component and/or CD28.

An effector cell can be engineered to contain a reporter such that engagement of the TCR complex and the CD28 results in detectable activation of the reporter and/or expression of the reporter.

An effector cell can be a lymphocyte. An effector cell can be a T-cell, e.g., HuT-78, CEM, Molt-4, primary T-cell, etc. In certain instances, an effector cell is a Jurkat T-cell.

In order to provide a useful readout for the bioassays provided herein, an effector cell can comprise a reporter gene. The reporter gene can encode a quantifiable gene product, for example, an enzyme or a bioluminescent protein. The reporter gene can encode, for example, a luciferase, a beta lactamase, CAT, SEAP, or a fluorescent protein.

The expression of the reporter gene can be under the control of a TCR-pathway-dependent promoter that is activated upon binding of both the TCR complex and CD28. In certain embodiments, the promoter is an IL-2 promoter.

An effector cell can comprise a Luciferase reporter driven by an IL-2 promoter.

As provided herein, co-engagement of the TCR complex and CD28 can result in transcription from an IL-2 promoter. An IL-2 promoter can contain DNA binding sites for NFAT, NF-κB, and/or AP-1. In certain embodiments, an IL-2 promoter contains DNA binding sites for NFAT, NF-κB, and AP-1.

### IV. Soluble Proteins

The methods, compositions, and systems provided herein can be used to assess the activity of soluble proteins comprising an Fc domain and a CD28-binding domain. The soluble protein can be, for example, an Fc fusion protein or an anti-CD28 antibody.

The Fc domain can be the Fc domain of an IgG. The Fc domain can be the Fc domain of a human immunoglobulin. In certain aspects, the Fc domain is a human IgG Fc domain. In certain aspects, the Fc domain is a human IgG1 Fc domain. In certain aspects, the human IgG1 Fc domain comprises, consists essentially of, or consists of the amino acid sequence set forth in SEQ ID NO:3.

The CD28-binding domain can be, for example CD80 or a CD28-binding fragment thereof. CD28-binding fragments of CD80 can comprise the V-like domain of CD80. The V-like domain occurs in SEQ ID NO:1 from amino acid 1 to 101. The CD28-binding domain can be CD86 or a CD28-binding fragment thereof. CD28-binding fragments of CD86 can comprise the V-like domain of CD86. The CD28-binding domain can be the antigen-binding domain of an anti-CD28 antibody.

In certain instances, the CD28-binding domain can be a CD80 extracellular domain (ECD). The CD80 ECD can, for example, be a human CD80 ECD. In certain aspects, the human CD80 ECD comprises, consists essentially of, or consists of the amino acid sequence set forth in SEQ ID NO:1.

The Fc domain and CD28-binding domain can be directly linked such that the N-terminal amino acid of the Fc domain follows the C-terminal amino acid of the CD28-binding domain (e.g., CD80 ECD). In certain aspects, the CD28-binding domain (e.g., CD80 ECD) and the Fc domain are translated as a single polypeptide from a coding sequence that encodes both the CD28-binding domain (e.g., CD80 ECD) and the Fc domain. In certain aspects, the Fc domain is directly fused to the carboxy-terminus of the CD28-binding domain (e.g., CD80 ECD).

In certain aspects, the soluble fusion protein comprises a human CD80 ECD and a human IgG1 Fc domain. In certain aspects, the fusion protein comprises, consists essentially of, or consists of the amino acid sequence set forth in SEQ ID NO:5.

CD80 ECD-Fc fusion proteins can, depending on how they are produced, have different levels of particular glycosylation modifications. For example, a CD80 ECD-Fc fusion protein can have different amounts of sialic acid (SA) residues.

In certain aspects, a CD80 ECD-Fc fusion protein (e.g., comprising SEQ ID NO:5) comprises 10 to 60 molecules of SA. In certain aspects, a CD80 ECD-Fc fusion protein (e.g., comprising SEQ ID NO:5) comprises 15 to 60 molecules of SA. In certain aspects, a CD80 ECD-Fc fusion protein (e.g., comprising SEQ ID NO:5) comprises 10 to 40 molecules of SA. In certain aspects, a CD80 ECD-Fc fusion protein (e.g., comprising SEQ ID NO:5) comprises 15 to 30 molecules of SA. In certain aspects, a CD80-ECD-Fc fusion protein (e.g., comprising SEQ ID NO:5) comprises 15 to 25 molecules of SA. In certain aspects, a CD80 ECD-Fc fusion protein (e.g., comprising SEQ ID NO:5) comprises 20 to 40 molecules of SA. In certain aspects, a CD80 ECD-Fc fusion protein (e.g., comprising SEQ ID NO:5) comprises 20 to 30 molecules of SA. In certain aspects, a CD80 ECD-Fc fusion protein (e.g., comprising SEQ ID NO:5) comprises 30 to 40 molecules of SA. In certain aspects, a CD80 ECD-Fc fusion protein (e.g., comprising SEQ ID NO:5) comprises 10, 15, 20, 25, 30, 35, or 40 molecules of SA. In certain aspects, a CD80 ECD-Fc fusion protein (e.g., comprising SEQ ID NO:5) comprises at least 15 molecules of SA. In certain aspects, a CD80 ECD-Fc fusion protein (e.g., comprising SEQ ID NO:5) comprises at least 20 molecules of SA. In certain aspects, a CD80 ECD-Fc fusion protein (e.g., comprising SEQ ID NO:5) comprises at least 25 molecules of SA. In certain aspects, a CD80 ECD-Fc fusion protein (e.g., comprising SEQ ID NO:5) comprises at least 30 molecules of SA. In certain aspects, a CD80 ECD-Fc fusion protein (e.g., comprising SEQ ID NO:5) comprises at least 35 molecules of SA. In certain aspects, a CD80 ECD-Fc fusion protein (e.g., comprising SEQ ID NO:5) comprises at least 40 molecules of SA.

### V. Compositions, Systems, and Methods for Assaying the Activity of Fc-Containing Proteins

Also provided herein are cell-based assay systems or compositions to assay the activity of proteins that contain Fc domains, such as Fc fusion proteins or antibodies. Such systems or compositions can comprise any activator cell provided herein and any effector cell provided herein. Described herein are “two-cell” assay systems that more accurately recapitulate in vivo conditions compared to other cell-based assay systems. Components on the surface of both the activator and effector cells participate in both the activation of the TCR by the TCR activator and the co-stimulation of CD28 by the Fc-containing protein, providing a more robust indication of T-cell co-stimulation pathway engagement.

The activator cell and the effector cell in the system or composition can be such that, upon interaction of a soluble protein comprising an Fc domain and a CD28-binding domain (e.g., a CD80 ECD-Fc fusion protein) with (i) the Fc receptor on the activator cell and (ii) the CD28 on the effector cell, the TCR complex activator (on the activator cell) activates the TCR complex (on the effector cell), and the CD28-binding domain (of the soluble protein) activates the CD28 signaling pathway via CD28 (on the effector cell).

Binding of the soluble protein to the Fc receptor on the activator cell and the CD28 on the effector cell can increase the signal from the reporter in the effector cell as compared to the signal in the presence of only the activator cell and/or in the presence of only the soluble protein.

The system or composition can comprise, for example, about 40,000 to about 55,000 activator cells or about 40,000 to about 50,000 activator cells. In certain instances, the system or composition comprises about 50,000 activator cells. In certain instances, the number of activator cells, as indicated above, are present in a volume of 100 μl.

The system or composition can comprise, for example, a concentration of activator cells that is about 0.4 million to about 0.55 million activator cells/mL or about 0.4 million to about 0.5 million activator cells/mL. In certain instances, the system or composition comprises about 0.5 million activator cells/mL.

The system or composition can comprise, for example, about 50,000 to about 200,000 effector cells. The system or composition can comprise, for example, about 100,000 effector cells. In certain instances, the number of effector cells, as indicated above, are present in a volume of 100 μl.

The system or composition can comprise, for example, a concentration of effector cells that is about 0.5 million to about 2 million effector cells/mL. In certain instances, the system or composition comprises about 1 million effector cells/mL.

In certain instances, the system or composition comprises a ratio of activator cells to effector cells that is from about 1:4 to about 1:1. In certain instances, the system or composition comprises a ratio of activator cells to effector cells that is from about 1:4 to about 3:4. In certain instances, the system or composition comprises a ratio of activator cells to effector cells that is about 1:2.

The system or composition can also comprise a soluble protein comprising an Fc domain and a CD28-binding domain (e.g., a CD80 ECD-Fc fusion protein). In certain instances of the systems or compositions provided herein, the soluble protein is present at a concentration of about 1 ng/mL to about 5000 ng/mL. In certain instances, the soluble protein is present at a concentration of about 1 ng/mL to about 3000 ng/mL. In certain instances, the soluble protein is present at a concentration of about 10, 15, 20, 25, 30, 35, 40, 45, 50, 55, 60, 65, 70, 75, 80, 85, 90, 95, or 100 ng/mL. In certain embodiments, the soluble protein is present at any of the above concentrations in a volume of 100 μl.

The system or composition can also comprise the above three components—activator cells, effector cells, and soluble protein comprising an Fc domain and a CD28-binding domain - in any combination of the amounts, concentrations, volumes, and ratios set forth above. In a non-limiting example, a system or composition may comprise about 0.5 million activator cells/mL; about 1 million effector cells/mL; and a soluble protein at a concentration of 10, 20, 30, 40, 50, 60, 70, 80, 90, or 100 ng/mL. In certain embodiments, such a system or composition may comprise about 50,000 activator cells at a concentration of 0.5 million activator cells/mL; about 100,000 effector cells at a concentration of about 1 million effector cells/mL; and a soluble protein at a concentration of 10, 20, 30, 40, 50, 60, 70, 80, 90, or 100 ng/mL; in a volume of 100 μl.

Provided herein are also methods of using the above systems and compositions provided herein. For example, in some embodiments, a method comprises incubating an activator cell provided herein and an effector cell provided herein with a soluble protein comprising an Fc domain and a CD28-binding domain and detecting a signal from the reporter gene in the effector cell.

The activator cell, effector cell, and soluble protein can be incubated together in the presence of a reporter assay buffer such as IL-2 Luciferase Reporter Assay Buffer of Promega's T Cell Activation Bioassay (catalog 01651). Thus, for example, activator cell diluted in reporter buffer, soluble protein, and effector cell diluted in reporter buffer can be mixed and incubated together. Alternatively, activator cell, soluble protein, effector cell, and reporter buffer can be mixed and incubated together.

The incubation can occur at a temperature of 35° C. to 39° C. In certain aspects of the methods provided herein, the incubation occurs at a temperature of about 37° C.

In certain aspects of the methods provided herein, the activator cells, effector cells, and soluble protein are incubated together for about 15 to about 25 hours, or for about 18 to about 22 hours.

After the incubation is complete, a signal from the reporter gene in the effector cell can be detected. A luciferase reporter signal can be detected, for example, using Bio-Glo™ Luciferase Assay Substrate of Promega's T Cell Activation Bioassay (catalog 01651). The Bio-Glo™ Luciferase Assay Substrate can be added to the composition or system comprising the activator cell, effector cell, and soluble protein for about 5 to about 15 minutes at room temperature before the resulting luminescent signal is detected.

The signal generated from the reporter gene in the presence of the soluble protein can be compared to the signal generated in the absence of the soluble protein and/or to the signal generated by a reference standard (e.g., a characterized sample of the soluble protein). The signal generated in the presence of the soluble protein can be significantly increased as compared to the signal generated in the absence of the soluble protein and/or compared to the signal generated in the absence of the activator cell.

In addition to or instead of comparing the signal generated from the reporter gene in the absence of an activator cell or in the absence of a soluble protein, the signal generated in the presence of an activator cell, an effector cell, and a soluble protein can be compared to the signal generated in the presence of an activator cell, an effector cell, and a reference standard.

### EXAMPLES

### Example 1

### Two-Cell Bioassay for Characterization of CD80 ECD-Fc

Different assay formats were explored to test the activity of a CD80 ECD-Fc fusion protein (SEQ ID NO:5). For example, “one-cell” bioassays in which reagents were immobilized on plates were attempted but did not yield robust and reproducible results. After exploring various alternatives, a two-cell bioassay was ultimately developed to test the activity of a CD80 ECD-Fc fusion protein (SEQ ID NO:5). In this assay, activator cells expressing an OKT3 scFv (SEQ ID NO:6) and a “tailless” form of CD64 (SEQ ID NO:7) were used. OKT3 scFv (referred to as “OKT3” in this Example) is a monoclonal antibody fragment that binds to CD3 in the TCR complex, and the tailless form of CD64 (referred to as “CD64” in this Example) is an Fc-gamma receptor in which the CD64 extracellular domain is fused to the CD32 transmembrane domain. The construction of these activator cells and their properties are described below in Examples 2-5.

In the two-cell bioassay, a vial of frozen activator cells (approximately 10′ cells/vial) were transferred into a 15 mL conical tube with 9 mL of IL-2 Luciferase Reporter Assay Buffer (4 ml of FBS (Promega's cat 0121A) to RPMI 1640 Medium (Promeg's cat #G708A) to a final concentration of 10%) and mixed well by inverting. The concentration of this cell dilution is about 0.6-1.4×10⁶ cells/mL. Cell count and viability are confirmed by analyzing 0.5 mL of cell suspension using Vi-CELL or hemocytometer by trypan blue exclusion. 50 μl of the cell dilution was pipetted into each well of an assay plate and placed onto a Coolsink® XT96F device for temperature uniformity distribution among wells.

Serial dilutions of CD80 ECD-Fc fusion protein were added in 25 μl aliquots to the wells to a final concentration of ˜1 ng/ml to 3 μg/ml.

TCR/CD3 Effector Cells (Promega's cat #J129A) were used in the assay. These effector cells express the TCR complex (including CD3 as a component) and CD28 on their surface, and they also contain a reporter gene in which luciferase expression is under control of the IL-2 promoter. The effector cells were thawed, and 0.8 mL of the cell suspension was transferred to a 15 mL conical tube with 3.2 mL of IL-2 Luciferase Reporter Assay Buffer and mixed by inversion. The concentration of this cell dilution is about 4×10⁶ cell/mL. Aliquots of the effector cell dilution (25 μl each) were added to each of the wells, and the plates were covered with a lid and incubated for 18-22 hours in a humidified cell culture incubator (37° C. and 5% CO2).

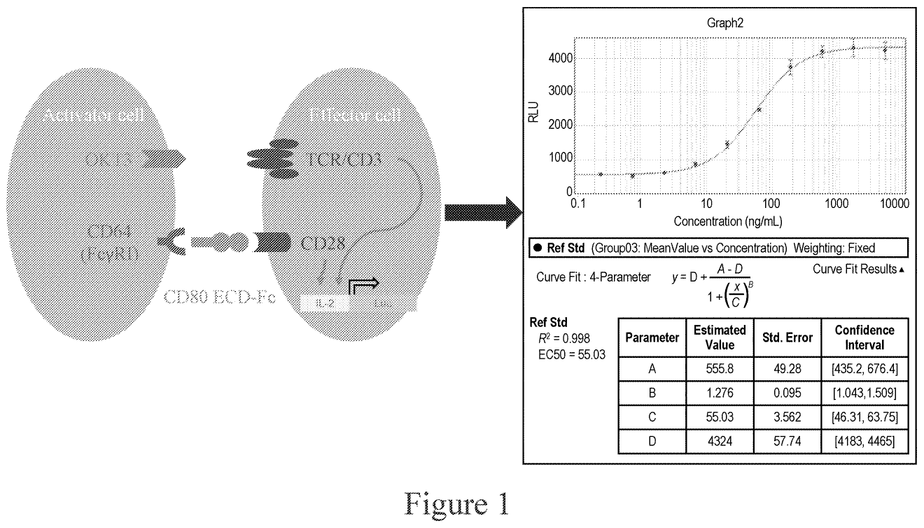

During incubation the Fc portion of the CD80-ECD fusion protein binds to CD64 expressed on the surface of the activator cells, and the CD80-ECD portion of the fusion protein binds to CD28 expressed on the surface of the effector cells, In parallel, OKT3 binds CD3 in the TCR complex expressed on the surface of the effector cells. Together, these interactions trigger activation of the IL-2 promoter-driven reporter construct, thereby driving an increase in detectable luciferase expression. The amount of luciferase signal is correlated with the amount of CD80 ECD-Fc fusion protein at each dilution by a dose response curve. (See FIG. 1.)

The signaling was quantified by the addition of Bio-Glo™ Luciferase Assay reagents. Bio-Glo™ Luciferase Assay Buffer (Promega's Cat. # G719A) was transferred into an amber bottle containing the Bio-Glo™ Luciferase Assay Substrate (Promega's Cat. # G720A) at room temperature and mixed by inversion until the Substrate was thoroughly dissolved and stored for 1.5 to 6 hours. The assay plates were removed from the incubator and equilibrated to room temperature for 15-20 minutes. Then 100 μl of the Bio-Glo™ reagent was added to each well of the assay plate, and the plates were shaken at 250 RPM for 5-7 minutes at room temperature. The plates were then read within 15 minutes after the incubation using a plate reader with glow-type luminescence reading. Exemplary results in the form of a dose response curve are shown in FIG. 1, panel on the right. The parameters listed underneath the curve (A, B, C, and D) are defined as follows: A is the Relative Luminscence Unit (RLU) value corresponding to the lower asymptote; B is a slope-like parameter (Hill Slope) of the dose response curve between the lower and upper asymptotes of the sigmoidal curve; C is the concentration of the fusion protein in ng/ml at which response is 50% of maximum (EC₅₀); and D is the RLU value corresponding to the upper asymptote. The EC₅₀ of the CD80-Fc fusion protein is found at the inflection point of the curve and was determined to be 55.03 ng/ml.

The two cell assay can be performed with a CD80-Fc fusion protein reference standard, as described above, and a test sample to determine the relative potency of the test sample. The A, B, and D parameters of the test sample curve is constrained to the A, B, and D parameters of the reference standard, and the relative potency is calculated as follows:

Potency Ratio=C Value of Reference Standard/C Value of Test Sample

where C=EC₅₀ (inflection point)

The potency ratio is multiplied by 100 to calculate the % Relative Potency. A test sample may be considered acceptable if the Relative Potency is between 50-150%.

### Example 2

### Construction of Activator Cell

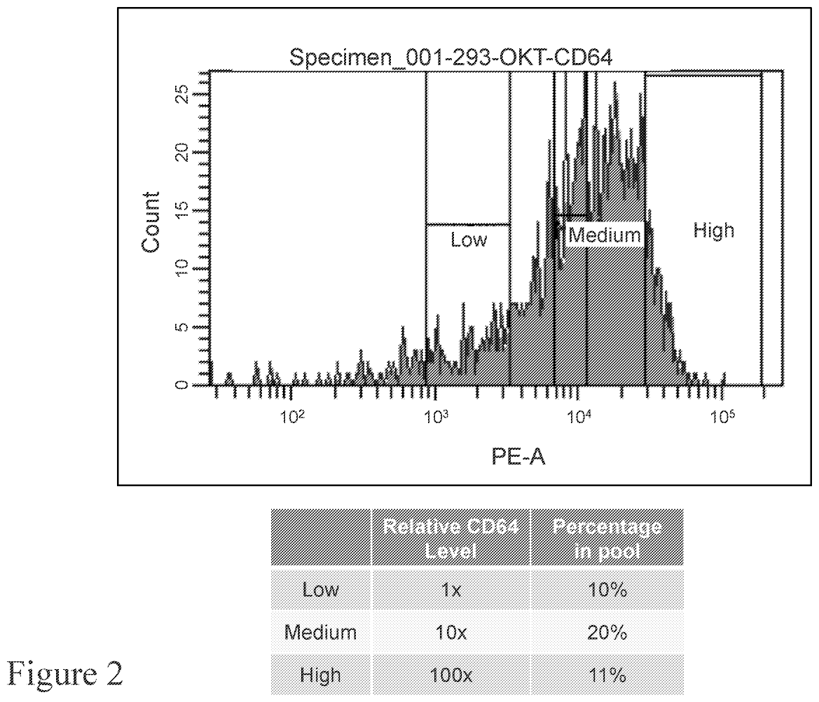

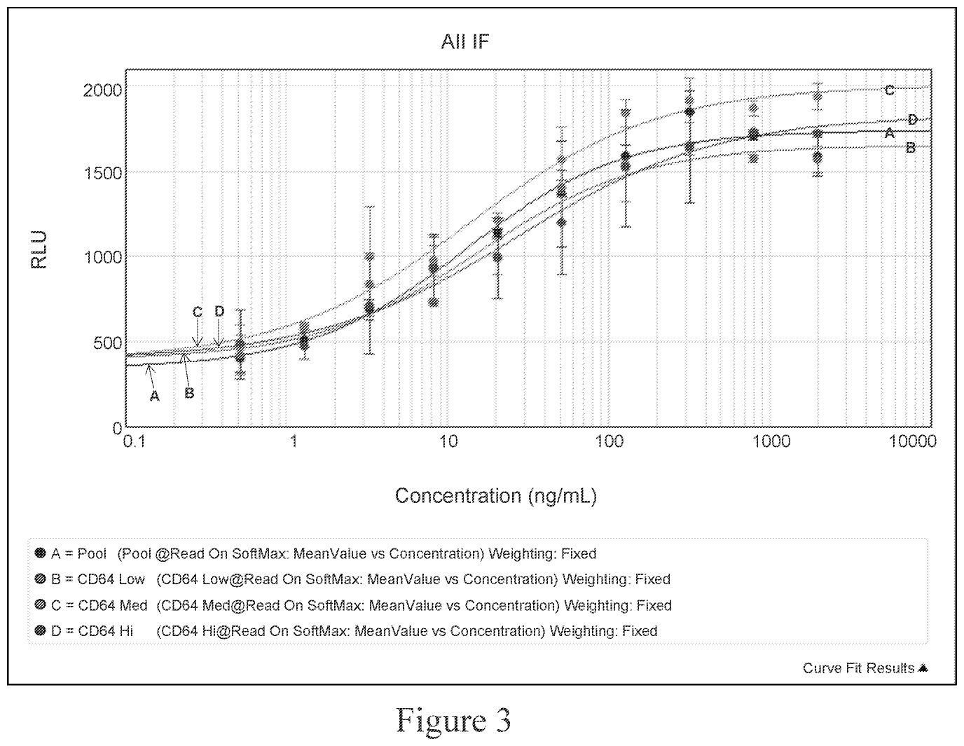

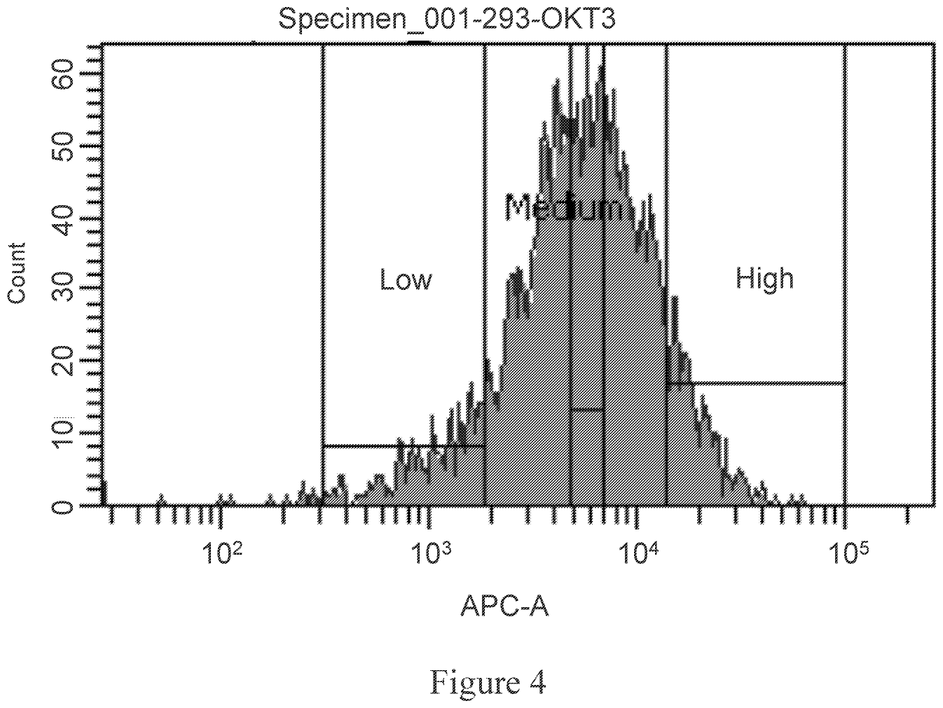

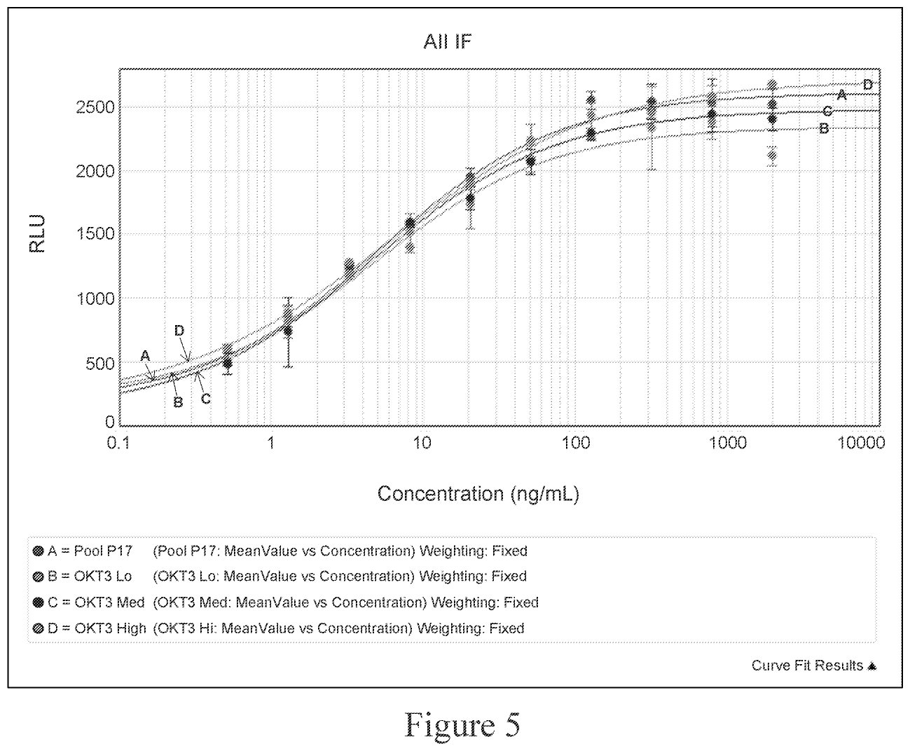

Human embryonic kidney HEK293 cells were used to create the activator cells. The HEK293 cells were transfected to express OKT3 scFv (SEQ ID NO:6) and a “tailless” form of CD64 (SEQ ID NO:7). As discussed above, OKT3 scFv (referred to as “OKT3” in this Example) is a monoclonal antibody fragment that binds to CD3 in the TCR complex, and the tailless form of CD64 (referred to as “CD64” in this Example) is an Fc-gamma receptor in which the CD64 extracellular domain is fused to the CD32 transmembrane domain. (See FIG. 1.) A range of OKT3 and CD64 levels were observed after transfection. Cells were sorted by FACS based on CD64 and OKT3 expression levels to determine the levels that result in optimal responses to immune modulators such as a CD80-ECD fusion protein using an assay similar to that described in Example 1. Sorted cells having low expression of OKT3 (relative to the range of OKT3 expression levels that were observed) and high expression of CD64 (relative to the range of CD64 expression levels that were observed) performed well in the assay and were selected. The cells were then further sorted by FACS based on CD64 (FIG. 2) and OKT3 (FIG. 4) expression levels. OKT3-low cells were selected (with CD64 levels not significantly affecting assay performance in this further round of sorting). The assay results after this round of sorting are shown in FIG. 3 (CD64) and FIG. 5 (OKT3) and in the tables below.

### CD64 (See FIG. 3.)

| | A | B | C | D | D/A |
|---|---|---|---|---|---|
| Pool | 339.2 | 0.884 | 12.93 | 1740 | 5.1 |
| Low | 393.1 | 0.858 | 15.50 | 1652 | 4.2 |
| Medium | 382.6 | 0.740 | 13.61 | 2002 | 5.2 |
| High | 390.4 | 0.696 | 26.67 | 1828 | 4.7 |

### OKT3 (See FIG. 5)

| | A | B | C | D |
|---|---|---|---|---|
| Pool | 171.1 | 0.733 | 5.306 | 2611 |
| Low | 211.0 | 0.743 | 4.758 | 2343 |
| Medium | 113.0 | 0.721 | 4.623 | 2480 |
| High | 172.8 | 0.625 | 5.904 | 2710 |

Single clone selection was pursued. A single clone referred to as “M1,” having OKT3-low and CD64-medium expression, performed optimally in the assay and was selected to establish the activator cell line. M1 demonstrated high cell viability (94-97%), and a desirable signal to background ratio (e.g., 6.1), EC50 range (49-65 (12% RSD)), and growth rate (about 24 hours).

### Example 3

### Evaluation of Activator Cell Number

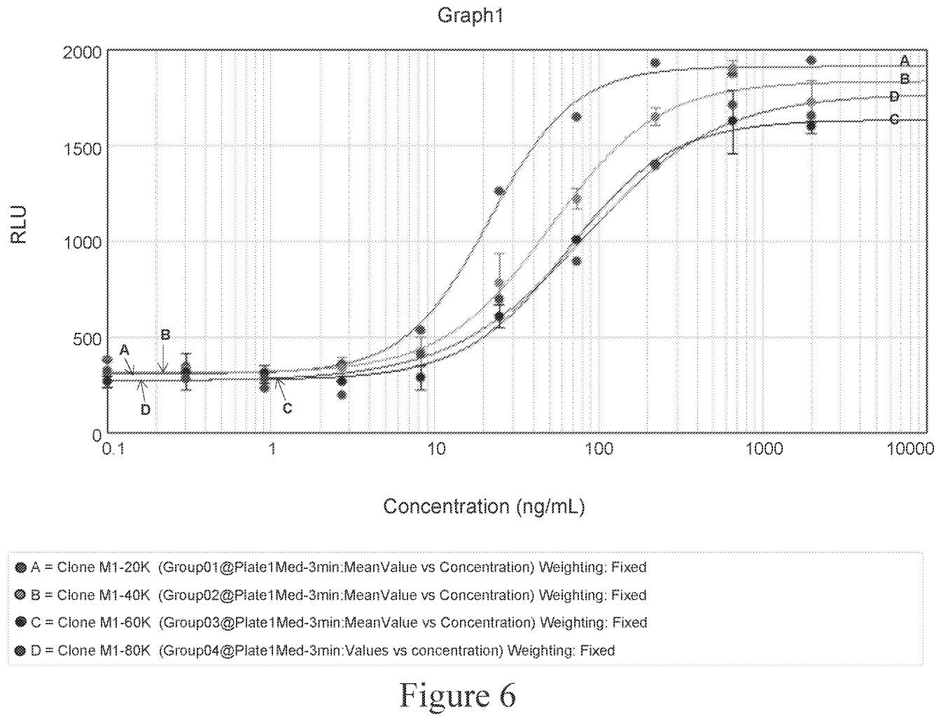

To optimize the seeding density of activator cells, varying numbers of M1 activator cells were plated in different wells (20K, 40K, 60K, or 80K per well), and the two-cell bioassay essentially as described above in Example 1 was performed. The results are shown in FIG. 6 and in the table below.

### Activator Cell Seed Density (See FIG. 6.)

| Seed Density | | | | | |
|---|---|---|---|---|---|
| (K cells per well) | A | B | C | D | D/A |
| 20 | 307 | 1.7 | 22 | 1909 | 6.2 |
| 40 | 310 | 1.3 | 51 | 1833 | 5.9 |
| 60 | 273 | 1.4 | 65 | 1631 | 6.0 |
| 80 | 269 | 1.1 | 82 | 1767 | 6.6 |

These results, along with other titration experiments, demonstrated that the optimal activation cell seeding density is 50 k cells/well.

### Example 4

### Cell Line Stability

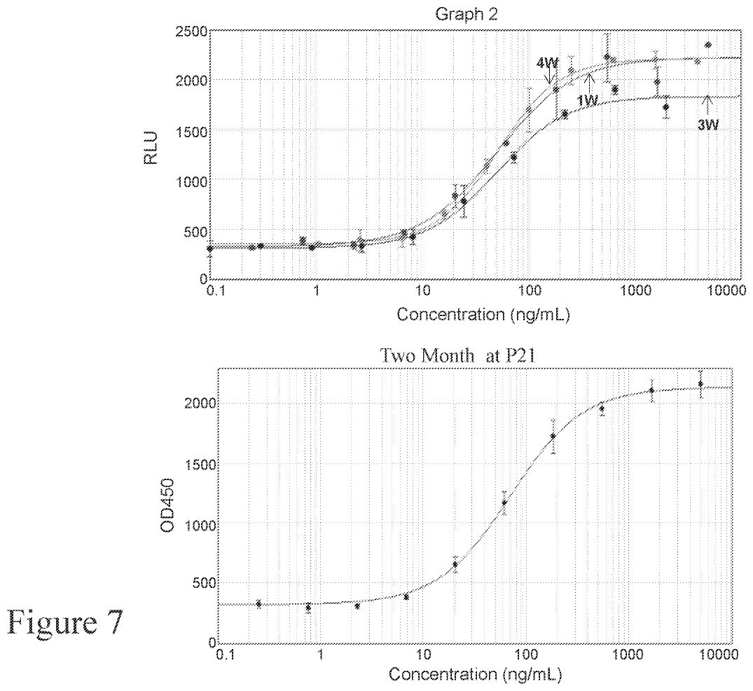

M1 activator cells were evaluated over time as the cells were continuously cultured. At various time points, the cells were used in a two-cell bioassay as discussed in Example 1 with increasing concentrations of CD80 ECD-Fc. Exemplary results with cells continuously cultured for 1, 3, and 4 weeks are shown in FIG. 7 and in the table below.

### Cell Stability (See FIG. 7)

| | A | B | C | D |
|---|---|---|---|---|
| 1 week | 325.8 | 1.206 | 50.68 | 2224 |
| 3 weeks | 310.1 | 1.320 | 50.55 | 1833 |
| 4 weeks | 352.4 | 1.498 | 51.43 | 2213 |

Similar luminescence (RLU) signals were detected across concentrations of CD80

ECD-Fc ranging from less than 1 ng/mL to over 1,000 ng/mL in cells that are cultured for 1, 3, or 4 weeks (FIG. 7 and table above). These results, along with additional experiments, show that activator cells can demonstrate stable assay performance up to 3 months.

### Example 5

### Activator Cells are Stable After Freezing and Thawing

In order to determine the feasibility of freezing, thawing, and then using the activator cell lines, activator cells were frozen in freezing medium (recovery medium +10% DMSO) in liquid nitrogen at Passage 15. The cells were later thawed into either assay medium (RPMI medium+10% FBS) or recovery medium (assay medium plus antibiotics) and used directly without any cell culture (“thaw and use” format) in a two-cell bioassay as discussed in Example 1. As a comparison, a vial of frozen activator cells from a cell bank were thawed and cultured over 16 passages (“continuously cultured”) prior to use in the two-cell bioassay.

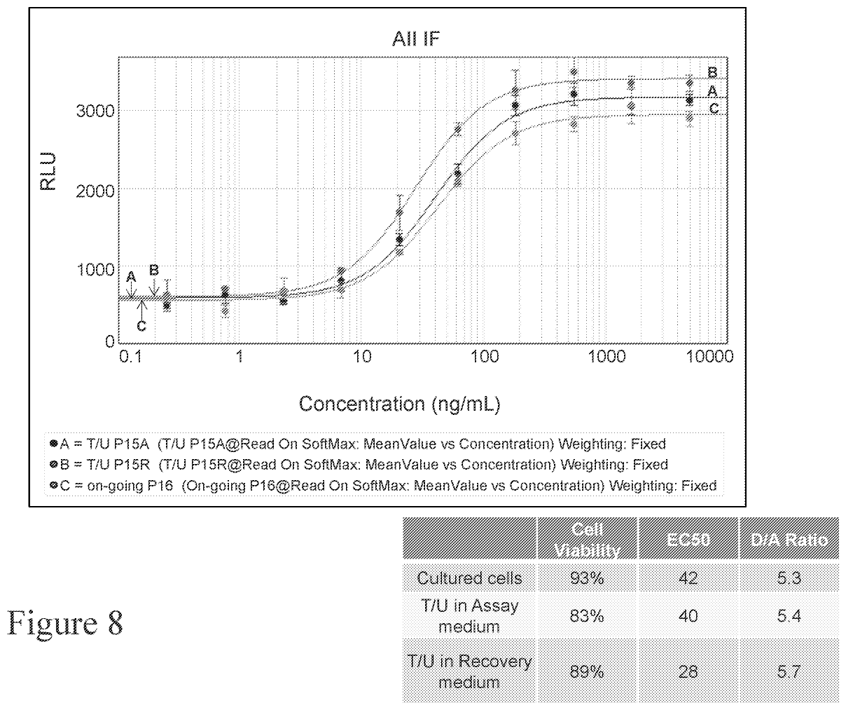

The results, shown in FIG. 8, demonstrate that the “thaw and use” activator cells (referred to as “T/U P15 A” (thawed into assay medium) or “T/U P15 R” (thawed into recovery medium) in FIG. 8) and the continuously cultured activator cells (referred to as “on-going P16” in FIG. 8) produced comparable results over increasing concentrations of CD80 ECD-Fc ranging from less than 1 ng/mL to over 1,000 ng/mL. Although there are some differences between the “T/U P15 A” and “T/U P15 R” cells, these differences are within the expected range of the assay during development. Moreover, the results show that the “thaw and use” format resulted in an appropriate D/A ratio (fold response) that is also within the expected EC50 range, and T/U cell viability was sufficient and comparable to that of the continuously cultured cells. In conclusion, the “thaw and use” format produces results that are consistent with a continuous culture approach, which is less convenient and takes longer to perform.

Moreover, the “thaw and use” activator cells were successfully used with frozen effector cells that were also thawed and used directly in the assay. The ability to obtain robust and reproducible results using both activator and effector cells in a “thaw and use” format provides significant advantages over the need to continuously culture cells. As mentioned above, continuously culturing cells results in increased costs and time and decreased convenience. In addition, continuously culturing cells over extended time periods may result in cell variability over time and therefore decrease the robustness of the assay.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

TABLE OF SEQUENCES
The table below provides a listing of certain sequences referenced herein

| SEQ. ID. NO. | Description | Sequence |
|---|---|---|
| 1 | Human CD80 | VIHVTKEVKEVATLSCGHNVSVEELAQTRIYWQKEKKMVLTMMSGDMN |
| | ECD sequence | IWPEYKNRTIFDITNNLSIVILALRPSDEGTYECVVLKYEKDAFKREH |
| | (without signal | LAEVTLSVKADFPTPSISDFEIPTSNIRRIICSTSGGFPEPHLSWLEN |
| | sequence) | GEELNAINTTVSQDPETELYAVSSKLDFNMTTNHSFMCLIKYGHLRVN |
| | | QTFNWNTTKQEHFPDN |
| 2 | Mouse CD80 | VDEQLSKSVKDKVLLPCRYNSPHEDESEDRIYWQKHDKVVLSVIAGKL |
| | ECD sequence | KVWPEYKNRTLYDNTTYSLIILGLVLSDRGTYSCVVQKKERGTYEVKH |
| | (without signal | LALVKLSIKADFSTPNITESGNPSADTKRITCFASGGFPKPRFSWLEN |
| | sequence) | GRELPGINTTISQDPESELYTISSQLDFNTTRNHTIKCLIKYGDAHVS |
| | | EDFTWEKPPEDPPDSKN |
| 3 | Fc human | EPKSSDKTHTCPPCPAPELLGGPSVFLFPPKPKDTLMISRTPEVTCVV |
| | IgG1 | VDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELT |
| | | KNQVSLTCLVKGFYPSDIAVEWESNGQPENNYKTTPPVLDSDGSFFLY |
| | | SKLTVDKSRWQQGNVFSCSVMHEALHNHYTQKSLSLSPGK |
| 4 | Mouse CD80 | VDEQLSKSVKDKVLLPCRYNSPHEDESEDRIYWQKHDKVVLSVIAGKL |
| | ECD mouse Fc | KVWPEYKNRTLYDNTTYSLIILGLVLSDRGTYSCVVQKKERGTYEVKH |
| | IgG2a (Fc | LALVKLSIKADFSTPNITESGNPSADTKRITCFASGGFPKPRFSWLEN |
| | portion | GRELPGINTTISQDPESELYTISSQLDFNTTRNHTIKCLIKYGDAHVS |
| | underlined) | EDFTWEKPPEDPPDSKNEPRGPTIKPCPPCKCPAPNLLGGPSVFIFPP |
| | | KIKDVLMISLSPIVTCVVVDVSEDDPDVOISWFVNNVEVHTAQTQTHR |
| | | EDYNSTLRVVSALPIQHQDWMSGKEFKCKVNNKDLPAPIERTISKPKG |
| | | SVRAPQVYVLPPPEEEMTKKQVTLTCMVTDFMPEDIYVEWTNNGKTEL |
| | | NYKNTEPVLDSDGSYFMYSKLRVEKKNWVERNSYSCSVVHEGLHNHHT |
| | | TKSFSRTPGK |
| 5 | Human CD 80 | VIHVTKEVKEVATLSCGHNVSVEELAQTRIYWQKEKKMVLTMMSGDMN |
| | ECD Human | IWPEYKNRTIFDITNNLSIVILALRPSDEGTYECVVLKYEKDAFKREH |
| | Fc IgG1 WT | LAEVTLSVKADFPTPSISDFEIPTSNIRRIICSTSGGFPEPHLSWLEN |
| | (Fc portion | GEELNAINTTVSQDPETELYAVSSKLDFNMTTNHSFMCLIKYGHLRVN |
| | underlined) | QTFNWNTTKQEHFPDNEPKSSDKTHTCPPCPAPELLGGPSVFLFPPKP |
| | | KDTLMISRTPEVTCVVVDVSHEDPEVKFNWYVDGVEVHNAKTKPREEQ |
| | | YNSTYRVVSVLTVLHQDWLNGKEYKCKVSNKALPAPIEKTISKAKGQP |
| | | REPQVYTLPPSRDELTKNQVSLTCLVKGFYPSDIAVEWESNGQPENNY |
| | | KTTPPVLDSDGSFFLYSKLTVDKSRWQQGNVFSCSVMHEALHNHYTQK |
| | | SLSLSPGK |
| 6 | CD3 antibody | AGVHSQVQLQQSGAELARPGASVKMSCKASGYTFTRYTMHWVKQRPGQ |
| | HB-okt3-pBNEW: | GLEWIGYINPSRGYTNYNQKFKDKATLTTDKSSSTAYMQLSSLTSEDS |
| | OKT3 VH | AVYYCARYYDDHYCLDYWGQGTTLTVSSGGGGSGGGGSGGGGSQIVLT |
| | leader_OKT3. | QSPAIMSASPGEKVTMTCSASSSVSYMNWYQQKSGTSPKRWIYDTSKL |
| | hc_(GGGGS)3 | ASGVPAHFRGSGSGTSYSLTISGMEAEDAATYYCQQWSSNPFTFGSGT |
| | _OKT3.1c_ | KLEINRGGGGSGGGGSMGIIVAVVIATAVAAIVAAVVALIYCRKEQKL |
| | (GGGGS)2_CD3 | ISEEDL |
| | 2 TM | |
| | domain_Myc | |
| | (without signal | |
| | sequence; Myc | |
| | sequence | |
| | underlined) | |
| 7 | CD64 ECD: | GQVDTTKAVITLQPPWVSVFQEETVTLHCEVLHLPGSSSTQWFLNGTA |
| | CD32 TM | TQTSTPSYRITSASVNDSGEYRCQRGLSGRSDPIQLEIHRGWLLLQVS |
| | (without signal | SRVFTEGEPLALRCHAWKDKLVYNVLYYRNGKAFKFFHWNSNLTILKT |
| | sequence) | NISHNGTYHCSGMGKHRYTSAGISVTVKELFPAPVLNASVTSPLLEGN |
| | | LVTLSCETKLLLQRPGLQLYFSFYMGSKTLRGRNTSSEYQILTARRED |
| | | SGLYWCEAATEDGNVLKRSPELELQVLGLQLPTPVWFHGGGGSGGGGS |
| | | MGIIVAVVIATAVAAIVAAVVALIYCRK |
| 8 | CD3 antibody | MERHWIFLLLLSVTAGVHSQVQLQQSGAELARPGASVKMSCKASGYTF |
| | HB-okt3-pBNEW: | TRYTMHWVKQRPGQGLEWIGYINPSRGYTNYNQKFKDKATLTTDKSSS |
| | OKT3 VH | TAYMQLSSLTSEDSAVYYCARYYDDHYCLDYWGQGTTLTVSSGGGGSG |
| | leader_OKT3. | GGGSGGGGSQIVLTQSPAIMSASPGEKVTMTCSASSSVSYMNWYQQKS |
| | hc_(GGGGS)3 | GTSPKRWIYDTSKLASGVPAHFRGSGSGTSYSLTISGMEAEDAATYYC |
| | _OKT3.1c_ | QQWSSNPFTFGSGTKLEINRGGGGSGGGGSMGIIVAVVIATAVAAIVA |
| | (GGGGS)2_CD3 | AVVALIYCRKEQKLISEEDL |
| | 2 TM | |
| | domain_Myc | |
| | (signal | |
| | sequence in | |
| | bold; Myc | |
| | sequence | |
| | underlined) | |
| 9 | CD64 ECD: | MWFLTTLLLWVPVDGQVDTTKAVITLQPPWVSVFQEETVTLHCEVLHL |
| | CD32 TM | PGSSSTQWFLNGTATQTSTPSYRITSASVNDSGEYRCQRGLSGRSDPI |
| | (signal | QLEIHRGWLLLQVSSRVFTEGEPLALRCHAWKDKLVYNVLYYRNGKAF |
| | sequence in | KFFHWNSNLTILKTNISHNGTYHCSGMGKHRYTSAGISVTVKELFPAP |
| | bold) | VLNASVTSPLLEGNLVTLSCETKLLLQRPGLQLYFSFYMGSKTLRGRN |
| | | TSSEYQILTARREDSGLYWCEAATEDGNVLKRSPELELQVLGLQLPTP |
| | | VWFHGGGGSGGGGSMGIIVAVVIATAVAAIVAAVVALIYCRK |

What is claimed is:

1. An engineered activator cell comprising (i) a T cell receptor (TCR) complex activator and (ii) and an Fc-gamma receptor or an Fc-binding fragment thereof.

2. The activator cell of claim 1, wherein the TCR complex activator is an anti-CD3 antibody, an antigen-binding fragment thereof, or major histocompatibility complex (MHC).

3. The activator cell of claim 1, wherein the TCR complex activator is an anti-CD3 antibody or antigen-binding fragment thereof.

4. The activator cell of claim 3, wherein the anti-CD3 antibody or antigen-binding fragment thereof binds to the epsilon subunit of CD3.

5. The activator cell of any one of claims 2-4, wherein the anti-CD3 antibody or antigen-binding fragment thereof is capable of inducing T cell activation.

6. The activator cell of any one of claims 2-5, wherein the anti-CD3 antibody or antigen-binding fragment thereof is OKT3 or an antigen-binding fragment thereof.

7. The activator cell of any one of claims 2-6, wherein the anti-CD3 antibody or antigen-binding fragment thereof comprises the amino acid sequence set forth in SEQ ID NO:6.

8. The activator cell of claim 7, wherein the anti-CD3 antibody or antigen-binding fragment thereof is produced from a protein comprising the amino acid sequence set forth in SEQ ID NO:8.

9. The activator cell of a claims 1-8, wherein the activator cell comprises a nucleic acid encoding the TCR complex activator, wherein the nucleic acid encoding the TCR complex activator is operably linked to a CAG promoter.

10. The activator cell of any one of claims 1-9, wherein the Fc-gamma receptor or Fc-binding fragment thereof is an IgG receptor or Fc-binding fragment thereof.

11. The activator cell of any one of claims 1-10, wherein the Fc-gamma receptor or Fc-binding fragment thereof is CD64 or an Fc-binding fragment thereof.

12. The activator cell of any one of claims 1-11, wherein the Fc-gamma receptor or Fc-binding fragment thereof comprises the amino acid sequence set forth in SEQ ID NO:7.

13. The activator cell of claim 12, wherein the Fc-gamma receptor or Fc-binding fragment thereof is produced from a protein comprising the amino acid sequence set forth in SEQ ID NO:9.

14. The activator cell of a claims 1-13, wherein the activator cell comprises a nucleic acid encoding the Fc-gamma receptor or Fc-binding fragment thereof, wherein the nucleic acid encoding the Fc-gamma receptor or Fc-binding fragment thereof is operably linked to a CAG promoter.

15. The activator cell of any one of claims 1-14, wherein the activator cell is a HEK293 cell.

16. The activator cell of any one of claims 1-15, wherein the activator cell is stable for at least 1 month.

17. The activator cell of claim 16, wherein the activator cell is stable for at least 2 months.

18. The activator cell of claim 17, wherein the activator cell is stable for at least 3 months.

19. A composition comprising a plurality of activator cells as in any one of claims 1-18.

20. The composition of claim 19, wherein the activator cells have at least 90% viability.

21. The composition of claim 19, wherein the activator cells have at least 94% viability.

22. The composition of claim 19, wherein the activator cells have about 94% to about 97% viability.

23. The composition of any one of claims 19-22, wherein the composition is frozen or wherein the composition is thawed after being previously frozen.

24. The composition of any one of claims 19-23, wherein the composition comprises about 5×10⁶-5×10⁷ activator cells, about 5×10⁶-1×10⁷ activator cells, or about 10⁷ activator cells.

25. A cell-based assay system comprising the activator cell of any one of claims 1-24 and an effector cell, wherein the effector cell comprises a T cell receptor (TCR) complex and CD28.

26. The system of claim 25, wherein upon interaction of a soluble protein comprising an Fc domain and a CD28-binding domain with (i) the Fc-gamma receptor or Fc-binding fragment thereof on the activator cell and (ii) the CD28 on the effector cell, the TCR complex activator activates the TCR complex and the CD28-binding domain activates the CD28 signaling pathway.

27. The system of claim 25 or 26, wherein the effector cell is a T-cell.

28. The system of claim 27, wherein the T-cell is a Jurkat, HuT-78, CEM, Molt-4, or primary T-cell.

29. The system of any one of claims 25-27, wherein the effector cell is a Jurkat T-cell.

30. The system of any one of claims 25-29, wherein the effector cell further comprises a reporter of TCR complex and CD28 activation.

31. The system of any one of claims 25-29, wherein the effector cell further comprises a reporter gene, the expression of which is under the control of a TCR-pathway-dependent promoter.

32. The system of claim 31, wherein the TCR-pathway-dependent promoter is an IL2 promoter.

33. The system of claim 31 or 32, wherein the reporter gene comprises a nucleic acid sequence encoding a bioluminescent protein.

34. The system of any one of claims 31-33, wherein the reporter gene comprises a nucleic acid sequence encoding a luciferase, a beta lactamase, CAT, SEAP, a fluorescent protein, or a quantifiable gene product.

35. The system of claim 34, wherein the reporter gene comprises a nucleic acid sequence encoding a luciferase.

36. The system of any one of claims 25-35, wherein the system comprises about 40,000 to about 55,000 activator cells.

37. The system of claim 36, wherein the system comprises about 40,000 to about 50,000 activator cells, optionally wherein the system comprises about 50,000 activator cells.

38. The system of any one of claims 25-37, wherein the system comprises about 0.4 million to about 0.55 million activator cells/mL.

39. The system of claim 38, wherein the system comprises about 0.4 million to about 0.5 million activator cells/mL, optionally wherein the system comprises about 0.5 million activator cells/mL.

40. The system of any one of claims 25-39, wherein the system comprises about 50,000 to about 200,000 effector cells, optionally wherein the system comprises about 100,000 effector cells.

41. The system of any one of claims 25-40, wherein the system comprises about 0.5 million to about 2 million effector cells/mL, optionally wherein the system comprises about 1 million effector cells/mL.

42. The system of any one of claims 25-41, wherein the ratio of activator cells to effector cells is about 1:4 to about 1:1.

43. The system of any one of claims 25-42 further comprising at least one soluble protein comprising an Fc domain and a CD28-binding domain.

44. The system of claim 43, wherein the soluble protein is present at a concentration of about 1 ng/mL to about 5000 ng/mL, optionally wherein the soluble protein is present at a concentration of about 1 ng/mL to about 3000 ng/mL.

45. A system comprising the activator cell of any one of claims 1-24 and at least one soluble protein comprising an Fc domain and a CD28-binding domain

46. The system of claim 45, wherein the soluble protein is present at a concentration of about 1 ng/mL to about 5000 ng/mL, optionally wherein the soluble protein is present at a concentration of about 1 ng/mL to about 3000 ng/mL.

47. A method comprising (a) incubating the activator cell and the effector cell in the system of any one of claims 30-42 and a soluble protein comprising an Fc domain and a CD28-binding domain and (b) detecting a signal from the reporter.

48. A method comprising (a) serially diluting a soluble protein comprising an Fc domain and a CD28-binding domain, (b) incubating the activator cell and the effector cell in the system of any one of claims 30-42 with the serially diluted soluble protein of (a), and (c) detecting a signal from the reporter.

49. The method of claim 47 or 48, further comprising comparing the signal from the reporter to the signal generated by a reference standard.

50. A method comprising (a) incubating the activator cell and the effector cell in the system of any one of claims 30-42, (b) detecting a signal from a reporter in the effector cell of (a), (c) incubating the activator cell and the effector cell in the system of any one of claims 30-42 with a soluble protein comprising an Fc receptor and a CD28-binding domain, (d) detecting a signal from the reporter in the effector cell of (c), and (e) comparing the signal of step (b) with the signal of step (d), wherein a gain of signal from step (b) to step (d) indicates biological activity of the soluble protein.

51. The method of any one of claims 47-50, wherein the amount of signal is proportional to the activity of the soluble protein present.

52. The method of any one of claims 47-51, wherein the incubating is for 18-22 hours.

53. The method of any one of claims 47-52, further comprising thawing the activator cells before the incubating.

54. The system or method of any one of claims 43-53, wherein the Fc domain is a human IgG1 Fc domain.

55. The system or method of any one of claims 43-54, wherein the Fc domain comprises the amino acid sequence set forth in SEQ ID NO:3.

56. The system or method of any one of claims 43-55, wherein the CD28-binding domain comprises CD80 or a fragment thereof, CD86 or a fragment thereof, or an anti-CD28 antibody or fragment thereof.

57. The system or method of any one of claims 43-55, wherein the CD28-binding domain comprises the extracellular domain of CD80.

58. The system or method of claim 57, wherein the extracellular domain of CD80 comprises the amino acid sequence set forth in SEQ ID NO:1.

59. The system or method of any one of claims 43-58, wherein the soluble protein comprises the amino acid sequence set forth in SEQ ID NO:5.

60. The system or method of claim 59, wherein the at least one soluble protein further comprises sialic acid (SA).

61. The system or method of claim 60, wherein the soluble protein comprises 15-60 moles of SA per mole protein.

62. The system or method of claim 60, wherein the soluble protein comprises 15-40 moles of SA per mole protein.

63. The system or method of claim 60, wherein the soluble protein comprises 15-30 moles of SA per mole protein.

64. The system or method of claim 60, wherein the soluble protein comprises 20-60 moles of SA per mole protein.

65. The system or method of claim 60, wherein the soluble protein comprises 20-30 moles of SA per mole protein.


SEQUENCE LISTING (9 sequences)

SEQ ID NO: 1 - protein, 208 aa, Artificial Sequence [sequence omitted]

SEQ ID NO: 2 - protein, 209 aa, Artificial Sequence
  Mouse CD80 ECD sequence
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKN

SEQ ID NO: 3 - protein, 232 aa, Artificial Sequence
  Fc human IgG1
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 442 aa, Artificial Sequence
  Mouse CD80 ECD mouse Fc IgG2a
VDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 5 - protein, 440 aa, Artificial Sequence
  Human CD80 ECD Human Fc IgG1 WT
VIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 294 aa, Artificial Sequence
  CD3 antibody HB-okt3-pBNEW OKT3 VH leader_OKT3.hc_
AGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSMGIIVAVVIATAVAAIVAAVVALIYCRKEQKLISEEDL

SEQ ID NO: 7 - protein, 316 aa, Artificial Sequence
  CD64 ECD CD32 TM
GQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHGGGGSGGGGSMGIIVAVVIATAVAAIVAAVVALIYCRK

SEQ ID NO: 8 - protein, 308 aa, Artificial Sequence
  CD3 antibody HB-okt3-pBNEW OKT3 VH
MERHWIFLLLLSVTAGVHSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGGGGSGGGGSMGIIVAVVIATAVAAIVAAVVALIYCRKEQKLISEEDL

SEQ ID NO: 9 - protein, 330 aa, Artificial Sequence
  CD64 ECD CD32 TM
MWFLTTLLLWVPVDGQVDTTKAVITLQPPWVSVFQEETVTLHCEVLHLPGSSSTQWFLNGTATQTSTPSYRITSASVNDSGEYRCQRGLSGRSDPIQLEIHRGWLLLQVSSRVFTEGEPLALRCHAWKDKLVYNVLYYRNGKAFKFFHWNSNLTILKTNISHNGTYHCSGMGKHRYTSAGISVTVKELFPAPVLNASVTSPLLEGNLVTLSCETKLLLQRPGLQLYFSFYMGSKTLRGRNTSSEYQILTARREDSGLYWCEAATEDGNVLKRSPELELQVLGLQLPTPVWFHGGGGSGGGGSMGIIVAVVIATAVAAIVAAVVALIYCRK